(19) United States
(12) Patent Application Publication
(10) Pub. No.: US 2026/0069758 A1
(43) Pub. Date: Mar. 12, 2026

(54) Apparatus for Applying Therapy to a Neurovascular Bundle

(71) Applicant: Wisconsin Alumni Research Foundation, Madison, WI (US)

(72) Inventors: Kip Ludwig, Middleton, WI (US); James Trevathan, Arena, WI (US); Rex Chin-Hao Chen, Waukesha, WI (US); Ricardo Sanchez, Madison, WI (US)

(21) Appl. No.: 18/828,019

(22) Filed: Sep. 9, 2024

(51) Int. Cl. A61M 1/00; A61M 5/158

(52) U.S. Cl. CPC A61M 1/77; A61M 5/1582; A61M 2205/3306; A61M 2205/332; A61M 2230/65

(57) ABSTRACT

A probe useful for introducing therapeutic materials between the neural sheath and contained neurovascular tissue provides a near field imaging device permitting final positioning of the probe to be conducted with submillimeter accuracy and a vacuum channel allowing the probe to be attached to the neural sheath to withdraw it and provide a defect through which therapeutic agents can be introduced.

### STATEMENT REGARDING FEDERALLY SPONSORED RESEARCH OR DEVELOPMENT

This invention was made with government support under EB029251 awarded by the National Institutes of Health and under OD025306 awarded by the National Institutes of Health. The government has certain rights in the invention.

### CROSS-REFERENCE TO RELATED APPLICATIONS

--

### BACKGROUND OF THE INVENTION

The present invention relates to therapeutic treatment of neuroanatomical targets and in particular to an apparatus for and method of providing improved access to the neuroanatomical targets through a neural sheath.

Nonsurgical access to neural structures is important for a variety of therapeutic or diagnostic activities including the placement of electrodes for neuromodulation or the introduction of therapeutic agents to the neurovascular anatomy. In many cases, it is desirable to introduce these therapeutic agents beneath the neural sheath into the paraneural space. In current practice, a delivery needle may be maneuvered to approach the neural structure and pierce the neural sheath as guided by fluoroscopy or ultrasound imaging. The close proximity of the neural sheath to the neurovascular tissue creates a potential for damage of the neurovascular tissue during this procedure.

### SUMMARY OF THE INVENTION

The present invention provides a percutaneous delivery needle that provides real-time, near field visualization of tissue near the tip of the delivery needle allowing precise positioning of the needle at the critical last millimeters of approach to the neural sheath. A vacuum channel allows the neural sheath to be engaged, pulled away from the neurovascular tissue so that an opening may be made in the neural sheath through which therapy can be introduced. The ability to visualize and manipulate the neural sheath greatly improves the ability to safely introduce therapeutic materials including drugs and electrodes.

More specifically, in one embodiment, the invention provides an apparatus for delivering therapy through a neural sheath and having a probe adapted for percutaneous insertion of a distal end into a patient along an axis. The probe supports a near field sensor having a sensor end positioned proximate to the distal end of the probe and operating to distinguish tissue types and distances with submillimeter resolution forward along the axis. The probe also supports a vacuum channel proximate to the distal end of the probe communicating with a vacuum system adapted to create a negative pressure for manipulating the neural sheath for the delivery of therapeutic material therethrough.

It is thus a feature of at least one embodiment of the invention to provide critical guidance for the final stages of positioning a probe against the neural sheath and an ability to manipulate that sheath to produce or assist in producing an opening through the sheath removed from contained neurovascular tissue.

In some embodiments, the probe may further include a dedicated delivery channel positioned to communicate with the defect for the delivery of the therapeutic material.

It is thus a feature of at least one embodiment of the invention to permit simultaneous or independent operation of the vacuum channel to stabilize the neural sheath without interference with delivery of therapeutic materials.

When a delivery channel is provided, the vacuum channel may surround the delivery channel.

It is thus a feature of at least one embodiment of the invention to permit a sealing off of the therapeutic channel during use.

The vacuum system may be adapted to create a negative pressure for drawing the neural sheath away from a contained neurovascular bundle.

It is thus a feature of at least one embodiment of the invention to provide an apparatus that can pull the neural sheath away from the neurovascular tissue to reduce interference with or damage to the neurovascular tissue during the opening of the neural sheath and/or introduction of therapeutic materials.

The probe may further include an electrode positioned near the vacuum inlet positioned to communicate electrically through the defect with nerves within the neural sheath.

It is thus a feature of at least one embodiment of the invention to provide an ability to confirm proper positioning along the neural sheath through electrical stimulation.

The near field sensor may be a polarization sensitive optical coherence tomographic sensor.

It is thus a feature of at least one embodiment of the invention to provide a near field sensor that can distinguish both tissue types and distances through use of polarized light for guidance of the distal end of the probe.

In some embodiments, the probe may include a stylet proximate to the vacuum inlet for piercing the nerve sheath.

It is thus a feature of at least one embodiment of the invention to provide a distinct mechanism for piercing the nerve sheath, for example, allowing more precise control of the opening size, cauterization, or the like.

In some embodiments, the probe may include an environmental sensor selected from the group consisting of an impedance sensor and a force sensor positioned for sensing outward from the distal end of the probe.

It is thus a feature of at least one embodiment of the invention to provide additional information about tissue type and locating the probe tip against the nerve sheath.

In some embodiments, the probe may further include a position sensor selected from the group consisting of an accelerometer and a gyroscope positioned at the distal end of the probe.

It is thus a feature of at least one embodiment of the invention to provide additional positional information that may augment or supplant external imaging.

These particular objects and advantages may apply to only some embodiments falling within the claims and thus do not define the scope of the invention.

### BRIEF DESCRIPTION OF THE DRAWINGS

FIG. 1 is a fragmentary perspective of a distal end of a probe according to the present invention and, in schematic, associated elements communicating with the probe including a computer and graphic display;

FIG. 2 is an elevational cross-section of a nerve sheath and contained neurovascular tissue showing an approach of the probe of FIG. 1 and an image on the graphic display of FIG. 1 depicting tissue layers and distances;

FIG. 3 is a figure similar to the cross-sectional view of FIG. 2 showing attachment of the distal end of the probe to the neural sheath using a negative pressure; and

FIG. 4 is a figure similar to FIGS. 2 and 3 showing a distention of the neural sheath by the probe and the creation of a defect through which therapeutic materials may enter into the paraneural space.

### DETAILED DESCRIPTION OF THE INVENTION

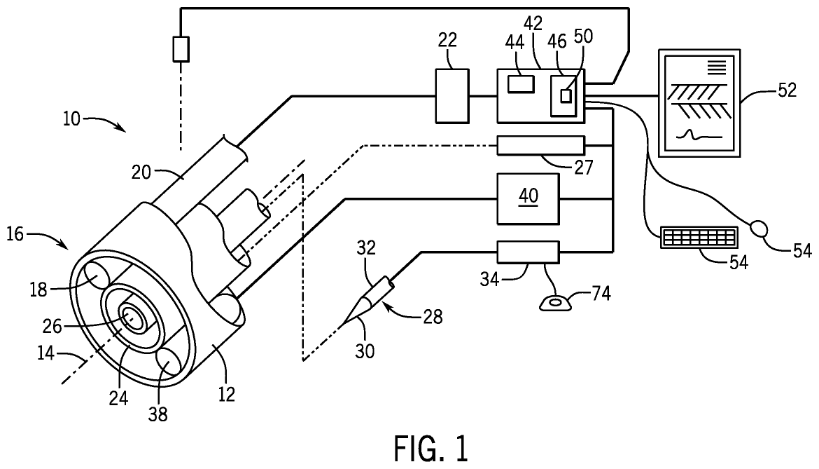

Referring to FIG. 1, a probe 10 according to one embodiment of the present invention may provide for an outer tubular shaft 12, for example, of stainless steel and sized for percutaneous insertion along an axis 14 through tissue of a patient. The length of the outer shaft is such as to permit the tubular shaft 12 to extend from a distal end 16 entering the patient tissue, to a location outside of the patient where the tubular shaft 12 may be manipulated by a physician or robotic device typically during imaging using ultrasound or x-ray fluoroscopy. In one nonlimiting example, an embodiment tubular shaft 12 may be an 18 gauge needle with a 1.2 mm outer diameter and a 1.07 mm inner diameter.

The tubular shaft 12 may support a near field sensor 18, for example, implemented by means of an optical fiber 20 communicating from the distal end of the tubular shaft 12 to sensor electronics 22 outside of the patient. The near field sensor 18 operates to provide imaging of tissue forward along the axis 14 from the distal end 16, the imaging distinguishing both tissue characteristics and distances from the near field sensor 18 with submillimeter resolution within a limited range typically less than a centimeter. In one embodiment, the near field sensor 18 may be a polarization-sensitive optical coherence tomographic sensor in which polarization sensitivity allows the distinguishing of tissues based on intensity and polarization of backscattered light, which is affected by the type of tissue and orientation of fibers or structures within the tissue.

A near field sensor 18 suitable for use with the present invention may be constructed according to the teachings of Benjamin J Vakoc, Seok-Hyun Yun, Johannes F De Boer, Guillermo J Tearney, and Brett E Bouma in Phase-resolved optical frequency domain imaging, Optics Express 13(14): 5483-5493, 2005 and the teachings of Benjamin J Vakoc, Ryan M Lanning, James A Tyrrell, Timothy P Padera, Lisa A Bartlett, Triantafyllos Stylianopoulos, Lance L Munn, Guillermo J Tearney, Dai Fukumura, Rakesh K Jain, and Brett E Bouma in Three-dimensional microscopy of the tumor microenvironment in vivo using optical frequency domain imaging, Nature Medicine, 15(10): 1219-1223, 2009 modified as taught by “Polarization-sensitive optical coherence tomography monitoring of percutaneous radiofrequency ablation in left atrium of living swine” found at https://www.nature.com/articles/s41598-021-03724-8, and “Needle guidance with Doppler-tracked polarization-sensitive optical coherence tomography” found at http://dx.doi.org/10.1117/1.JBO.28.10.102910, all hereby incorporated by reference.

The invention also contemplates the possibility of other near field sensors, for example, using principles of confocal microscopy, laser speckle imaging, photo acoustics, and high-frequency ultrasound.

The tubular shaft 12 may further contain a vacuum channel which may either be the unoccupied lumen of the tubular shaft 12 or an additional tubular conduit 24 positioned therein. This vacuum channel communicates with a vacuum pump 27 outside of the patient whose operation will be described below.

In one embodiment, an additional delivery channel 26 may also be provided, for example, positioned concentrically within the tubular conduit 24. The delivery channel 26 provides a dedicated path for the introduction of therapeutic materials as will be discussed below and may extend the full length of the tubular shaft 12 along the axis 14 to a position outside of the patient for the introduction of the therapeutic materials or other elements as may be desired.

In some embodiments, the invention may provide for a stylet 28 sized, for example, to fit slidably within the delivery channel 26 to help hold the delivery channel 26 open during insertion, assist in an opening of the neural sheath, and provide for electrical stimulation as will be discussed below. In this latter use, the stylet 28 may expose an electrically conductive sharpened tip 30 at the distal end 16 of the tubular shaft 12 and may be surrounded over a remainder of its length by an electrical insulator 32. The inner conductive shaft of the stylet 28 may then connect to stimulation and monitoring circuitry 34 for providing nerve stimulation as will be discussed further below.

In some embodiments, an additional sensor packet 38 may be positioned at the distal end 16 of the probe 10 including position sensors, for example, including but not necessarily limited to a MEMs gyroscope and multi-axis accelerometer, and/or environmental sensors including but not limited to an electrical impedance sensor and a force sensor. The electrical impedance sensor, for example, may sense the impedance of tissue forward from the distal end 16 of the probe 10 through capacitive, inductive, or resistive coupling. The force sensor, for example, may sense a force of tissue against the probe 10 during insertion, for example, to produce elasticity as will be discussed below. Signals from the sensor packet 38 may be processed by the data acquisition system 40.

The data acquisition system 40 may communicate with a computer 42 also communicating with the vacuum pump 27, the sensor electronics 22, the data acquisition system 40, and the stimulation and monitoring circuitry 34 to control or receive signals therefrom. The computer 42 may include one or more processors 44 communicating with an electronic memory 46 holding a stored program 50 executable to receive and display data from the sensor electronics, the data acquisition system 40 and the stimulation and monitoring circuitry 34 being on a graphic display 52. User input for the control of the stored program 50 may be provided by input devices 54 such as a mouse or keyboard also communicating with the computer 42.

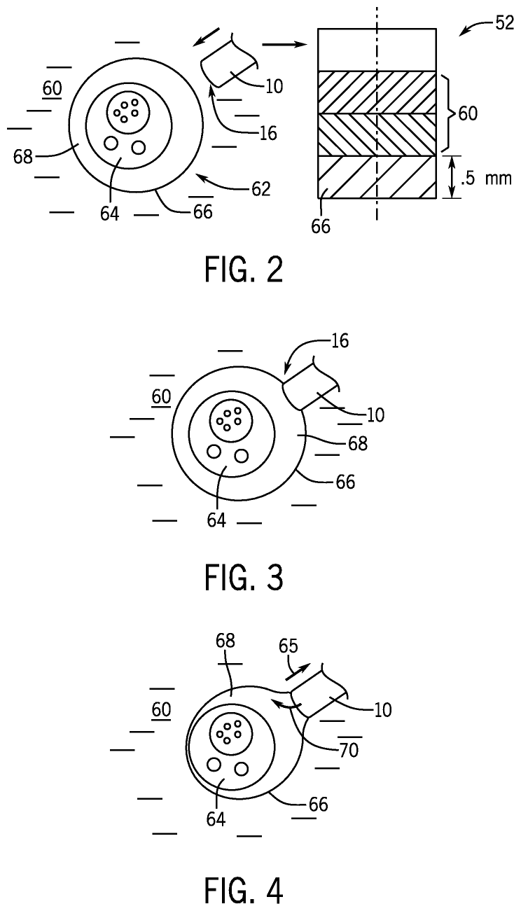

Referring now to FIG. 2, during use, the distal end 16 of the probe 10 may be inserted through tissue 60 of the patient to approach neural anatomy 62 of neurovascular tissue 64 held within a neural sheath 66 and separated from the neural sheath 66 by a paraneural space 68. This insertion of the probe 10 will normally be guided coarsely by an external imaging system 63 such as a fluoroscopic or ultrasound imaging device. The external imaging system 63 may also communicate with computer 42 and display 52 (or another computer and display) to provide information to the clinician guiding the distal end 16 of the probe 10 to a point where the distal end 16 is proximate to (for example, less than 1 cm removed from) the neural sheath 66.

At this point, the display 52 may provide an image obtained from the near field sensor 18 identifying both tissue types and distances forward from the distal end 16 with submillimeter accuracy. This image may be used to guide the distal end 16 into contact with the neural sheath 66 as indicated by FIG. 3. As noted above, the field sensor 18 can readily identify the transitions between planes of connective tissues, fat and muscle, or different layers of muscle tissue with changes in fiber orientation.

In some embodiments, during this guidance, data of the near field sensor 18 may be supplemented with impedance measurements using the sensor packet 38 or tissue elasticity measurements using the force sensor. These latter elasticity measurements may use the strain gauge to deduce the force of the probe as it is inserted through the tissue and make displacement measurements yielding elasticity. Examples of this technique are provided, for example, in Kennedy KM, Kennedy BF, McLaughlin RA, Sampson DD. Needle optical coherence elastography for tissue boundary detection. Opt Lett. 2012 June 15; 37(12): 2310-2. doi: 10.1364/OL.37.002310. PMID: 22739891, hereby incorporated by reference.

When the distal end 16 of the probe 10 is in contact with the neural sheath 66, as indicated in FIG. 3, the vacuum pump 27 may be activated to attach the neural sheath 66 to the distal end 16 so that the neural sheath 66 may be drawn away from the neurovascular tissue 64, as indicated by arrow 65, with a slight retraction of the probe 10. This action operates to increase the volume of the paraneural space 68 proximate to the distal end 16 to help protect the neurovascular tissue 64. The negative pressure from the vacuum pump 27 may be increased to create a defect in the neural sheath, or the defect may be created simply by the distention of the neural sheath 66, or the defect may be produced by insertion of the stylet 28 to puncture the neural sheath 66. For this purpose, the stylet 28 may include a stop limiting its distance of extension beyond the distal end 16. The resulting defect may have a diameter between 0.159 to 0.515 mm and may in some embodiments be cauterized by the stylet 28 through the application of electrical current to the tip 30 or other heating mechanism.

As so positioned, the stylet 28 may be used to excite the nerves of the neurovascular tissue 64 with respect to a skin electrode 74 (shown in FIG. 1) to establish proper location and communication by observation of muscle activity or communication with the patient. Alternatively, or in addition, the stylet 28 may be used during insertion to provide test nerve stimulation pulses to guide the location of the distal end 16.

The stylet 28 may then be removed from the delivery channel 26 and, per FIG. 4, therapeutic materials 70 introduced into the paraneural space 68 via the therapy delivery channel 26. The therapeutic materials 70 may include drugs, saline, and contrast media such as radio opaque die or the like, or may include so-called flexible electrodes, for example, silicone-metal-particle composites and flowable two-part conductive pre-polymers which may be injected through a needle and syringe into the paraneural space 68 adjacent to and surrounding neurovascular tissue 64 to quickly polymerize in vivo, as described in “An Injectable Neural Stimulation Electrode Made from an In-Body Curing Polymer/Metal Composite” found at at www.ncbi.nlm.nih.gov/pmc/articles/PMC10425988. Other example electrodes include those described in U.S. Pat. Nos. 8,676,309; 11,324,957; and in Dong, C., Carnicer-Lombarte, A., Bonafè, F. et al., Electrochemically actuated microelectrodes for minimally invasive peripheral nerve interfaces, Nat. Mater. 23, 969-976 (2024); A thin-film multichannel electrode for muscle recording and stimulation in neuroprosthetics applications, Silvia Muceli et al 2019 J. Neural Eng. 16 026035; all hereby incorporated by reference. These electrodes may be used as described in Update on Peripheral Nerve Electrodes for Closed-Loop Neuroprosthetics, Emil H. Rijnbeek et al. Front. Neurosci., 27 May 2018.

Alternatively, the electrode may be skeined or loosely coiled and twisted wires such as platinum iridium wires, for example, used in the Injectrode™ commercially available from “Recruitment of Primary Afferents by Dorsal Root Ganglion Stimulation using the Injectrode” found at www.ncbi.nlm.nih.gov/pmc/articles/PMC8502004/, and “Stimulation of the dorsal root ganglion using an Injectrode” found at //iopscience.iop.org/article/10.1088/1741-2552/ac2ffb, each of which is hereby incorporated by reference.

In both cases the implanted electrode may be used for stimulation or monitoring of the neurovascular tissue.

During use of the delivery channel 26, negative pressure may be maintained on the surrounding tubular conduit 24 to maintain a sealing of the probe 10 against the neural sheath 66.

The broadband nature of the near field sensor 18 allows one dimensional information to be acquired in front of probe 10 without any scanning. Alternatively, near field sensor 18 may use a scanning GRIN lens to scan one-dimensional or two-dimensional space. Combined with the depth profile provided by the PS-OCT, the PS-OCT system 54 allows for the reconstruction of 3D images. These embodiments may encompass four-dimensional imaging (including time) representations.

In one embodiment the information from the external imaging system 63 and from the probe 10 may be combined to provide the clinician with a comprehensive view of the progress of the probe 10 using techniques of sensor fusion.

It will be understood from this description that the probe 10 may be used to grab the relevant sheath of various neuroanatomical targets such as the nerve trunk, ganglia, nuclei, or other neural targets of interest, to pierce the sheath in a similar manner as described above. The probe 10 may also be used with other anatomical targets.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “an electronic computer” and “a processor” or “the microprocessor” and “the processor,” can be understood to include one or more of these devices that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

References to “a processor” should be understood to include electronic computers, microprocessors, microcontrollers, FPGA devices, ASIC devices and similar programmable or program defined electronic circuits and collections of such devices that can communicate in a stand-alone and/or a distributed environment(s), and can thus be configured to communicate via wired or wireless communications with other processors. Furthermore, references to memory, unless otherwise specified, can include one or more processor-readable and accessible memory elements and/or components that can be internal to the processor or external to the processor and accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S. C. 112(f) unless the words “means for” or “step for” are explicitly used in the particular claim.

We claim:

1. An apparatus for delivering therapy through a neural sheath comprising:
a probe adapted for percutaneous insertion of a distal end into a patient along an axis, the probe supporting:
a near field sensor having a sensor end positioned proximate to the distal end of the probe, the near field sensor distinguishing tissue types and distances with submillimeter resolution forward along the axis; and
a vacuum channel proximate to the distal end of the probe communicating with a vacuum system adapted to create a negative pressure for manipulating the neural sheath for a delivery of therapeutic material through a defect therein.

2. The apparatus of claim 1 further including a delivery channel positioned to communicate with the defect for the delivery of the therapeutic material.

3. The apparatus of claim 2 wherein the vacuum channel surrounds the delivery channel.

4. The apparatus of claim 1 wherein the vacuum system is adapted to create a negative pressure for drawing the neural sheath away from a contained neurovascular bundle.

5. The apparatus of claim 1 further including an electrode positioned near the vacuum inlet positioned to communicate electrically through the defect with nerves within the neural sheath.

6. The apparatus of claim 1 wherein the near field sensor is a polarization sensitive optical coherence tomographic sensor.

7. The apparatus of claim 1 further including a stylet proximate to the vacuum inlet for piercing the nerve sheath.

8. The apparatus of claim 1 further including an environmental sensor selected from the group consisting of an impedance sensor and a force sensor positioned for sensing outward from the distal end of the probe.

9. The apparatus of claim 1 further including a position sensor selected from the group consisting of an accelerometer and a gyroscope positioned at the distal end of the probe.

10. A method for delivering therapy through a neural sheath using a probe adapted for percutaneous insertion of a distal end into a patient along an axis, the probe supporting: a near field sensor having a sensor end positioned proximate to the distal end of the probe, the near field sensor distinguishing tissue types and distances with submillimeter resolution forward along the axis; and a vacuum channel proximate to the distal end of the probe communicating with a vacuum system adapted to create a negative pressure for producing a defect in the neural sheath for the delivery of therapeutic material therethrough, the method comprising:
(a) inserting the distal end of the probe through the tissue of the patient while monitoring output of the near field sensor to position the distal tip at the neural sheath;
(b) generating the negative pressure at the vacuum channel to manipulate the neural sheath for the introduction of therapeutic materials; and
(c) introducing therapeutic materials through a defect in the neural sheath.

11. The method of claim 10 wherein the probe further includes a delivery channel positioned to communicate with the defect for the delivery of the therapeutic material and delivering the therapeutic materials through the delivery channel.

12. The method of claim 11 wherein the vacuum channel surrounds the delivery channel.

13. The method of claim 10 including the step of retracting the probe to draw the neural sheath away from a contained neurovascular bundle as the probe is attached to the neural sheath by negative pressure.

14. The method of claim 10 wherein the probe further includes an electrode positioned near the vacuum inlet positioned to communicate electrically through the defect with nerves within the neural sheath and including stimulating nerves within the neural sheath using the electrode.

15. The method of claim 10 wherein the near field sensor is a polarization sensitive optical coherence tomographic sensor.

16. The method of claim 10 wherein the probe further includes a stylet proximate to the vacuum inlet and including creating the defect using the stylet while the neural sheath is retained with negative pressure through the vacuum channel.

17. The method of claim 10 wherein the probe further includes an environmental sensor selected from the group consisting of an impedance sensor and a force sensor positioned for sensing outward from the distal end of the probe and employing information from the environmental sensor to position the probe against the neural sheath.

18. The method of claim 10 further including a position sensor selected from the group consisting of an accelerometer and a gyroscope positioned at the distal end of the probe.
